(19) United States
(12) Patent Application Publication
(10) Pub. No.: US 2014/0370620 A1
(43) Pub. Date: Dec. 18, 2014

(54) MULTIMER TYPE DISCRIMINATION AND DETECTION METHOD FOR MULTIMER-FORMING POLYPEPTIDE

(71) Applicant: NANOENTEK, INC., Seoul (KR)

(72) Inventors: Chan Il Chung, Seoul (KR); Dae Sung Hur, Seoul (KR); Ho Young Kim, Seoul (KR); Jason Sung Kim, Rowland Heights, CA (US)

(73) Assignee: NANOENTEK, INC., Seoul (KR)

(21) Appl. No.: 14/369,896

(22) PCT Filed: Jan. 3, 2013
(86) PCT No.: PCT/KR2013/000025

§ 371 Date: Jun. 30, 2014

(30) Foreign Application Priority Data
Jan. 3, 2012 (KR) 10-2012-0000493

(51) Int. Cl. G01N 33/68

(52) U.S. Cl. CPC G01N 33/6896; G01N 2333/47
USPC 436/501; 422/82.05

(57) ABSTRACT

The present invention relates to a method for selectively detecting a multimer type multimer-forming polypeptide in a biological sample, the method comprising: (a) bringing the biological sample into contact with an agglutination reaction inducing agent to induce the formation of an aggregate in an analysis target, the agglutination reaction inducing agent being a particle in which a specific antibody is surface-bonded with the multimer-forming polypeptide; (b) obtaining an image with respect to the aggregate of step (a); and (c) analyzing a size or a shape of the aggregate by using the image. Step (a), step (b), or steps (a) and (b) are performed on a microchip having a microchannel. The image analysis is performed using a coefficient according to the size of the aggregate in a predetermined volume provided by the microchannel. In the case where the multimer type of multimer-forming polypeptide is present in the biological sample, the size of the aggregate is larger than the size of an aggregate of a monomer type control group. According to the present invention, unlike in a detection method using chemiluminescence immunoanalysis of the related art, an image with respect to an agglutination reaction target is obtained and then a size or a shape of an aggregate is analyzed so as to determine whether or not an analysis target is present in a biological sample and to determine the quantity of the analysis target. Also, it is possible to detect a multimer type multimer-forming polypeptide by just analyzing an image acquired from the sample so that the detection process is made more convenient and quick.

### TECHNICAL FIELD

This application claims priority to and the benefit of Korean Patent Application No. 10-2012-0000493 filed in the Korean Intellectual Property Office on Jan. 3, 2012, the entire contents of which are incorporated herein by reference.

The present invention relates to a method for selectively detecting a multimer type multimer-forming polypeptide.

### BACKGROUND ART

A multimerization of polypeptides constituting proteins has been generally known to be required for the function of proteins. However, multimeric forms often cause diseases or disorders in some proteins. In particular, a protein exists as a monomer in normal conditions and is converted into a multimer (or aggregated form) in abnormal conditions (e.g., conversion into a misfolding form). It has been known that proteins misfolded and ultimately aggregated (or accumulated), i.e., not having a functionally relevant conformation do not exhibit normal biological activity. If proteins are not correctly folded or do not maintain a correctly folded conformation, such a condition causes various types of biological malfunctions, and consequently various types of diseases (Massimo Stefani, et al., J. Mol. Med. 81:678-699(2003); and Radford S E, et al., Cell. 97:291-298(1999)). Protein molecules having incorrect conformations, that is, conformations that are different from those in normally functioning organisms cause many diseases in living organisms. The diseases or disorders associated with abnormal aggregation or misfolding of proteins, for example, include Alzheimer's disease, Creutzfeldt-Jakob disease, Spongiform encephalopathies, Parkinson's disease, Huntington's disease, Amyotrophic lateral sclerosis, Serpin deficiency, emphysema, cirrhosis, Type II Diabetes, primary systemic amyloidosis, secondary systemic amyloidosis Fronto-temporal dementias, senile systemic amyloidosis, familial amyloid polyneuropathy, hereditary cerebral amyloid angiopathy, and haemodialysis-related amyloidosis.

Alzheimer's disease (AD) is a degenerative brain disease that is a clinical characteristic of slowly progressive cognitive impairment and behavioral problems. A main pathological feature associated with this disease is that two water-insoluble protein materials aggregate and are precipitated in the hippocampus and cortex. Senile plaque (SP) composed of amyloid beta protein (Aβ) is accumulated outside neuronal cells, and nurofibriillary tangle (NFT) composed of hyperphosphorylated tau protein is accumulated inside the neuronal cells (Vetrivel K S et al., Neurology, 66:S69-73(2006), Lee V M et al., Neuron, 52:33-38(2006)).

One study of cell culture, animal models, and humans suggests that Aβ plays an important role in AD pathogenesis. Therefore, the primary target for the disease modifying strategy is Aβ. Aβ is mostly produced by neurocytes through the normal physiological processes in the central nervous system (CNS), and is relevant to activity of neuronal cells. Aβ secreted outside the neuronal cells is normally degraded, or is excreted outside the CNS to maintain homeostasis in the body. However, the Aβ level in brain tissues of AD patients is 100 to 1,000 times higher than the normal, and thus highly likely to form aggregates. This implies that Aβ increases in large quantities due to activation of its generation or deterioration of its excretion, but it has not yet been known to what extent each mechanism therefor is involved in AD pathogenesis (Fukumoto H et al., Arch Neurol, 59:1381-1389 (2002), Holtzman D M et al. Alzheimer Dis Assoc Disord, 17 (Suppl 2):S66-68 (2003)).

Progressive degenerative neurological diseases that account for most of senile dementia have increased social and economic burdens in an exponential manner with the increase of life expectancy. Thus, if clinical diagnosis of early AD becomes possible and timely treatment is made, the life quality of patients will be improved or the accompanying economic burden will be reduced. However, currently, since there is no decisive way to completely cure AD and symptomatic treatments have very limited efficacies, the initial clinical diagnosis is an important factor in the treatment procedure.

As a result of intensive research on early diagnoses of coagulation-related diseases, chemiluminescence-based image assay or immunoassay of induced aggregation of analytes has been mainly used. However, technologies for detecting analytes by analyzing sizes or shapes of aggregates from the image data of the aggregates have not yet been proposed. Therefore, the present inventors have researched methods for differentially detecting multimer-forming polypeptides causing various diseases, and then developed detecting methods which can help diagnose diseases at the early aggregation stage of polypeptides and develop therapeutic agents directly for the diseases.

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### DETAILED DESCRIPTION OF THE INVENTION

### Technical Problem

The present inventors have endeavored to develop a method for differentially detecting a multimeric form of a multimer-forming polypeptide. As a result, the present inventors have developed a method for detecting an analyte by obtaining an image of an aggregation reaction material and then analyzing sizes or shapes of aggregates, unlike the method for detecting an analyte by inducing an aggregation reaction of the analyte and employing chemiluminescence-based immunoassay. The method of the present invention can detect a multimeric form of a multimer-forming polypeptide through only an image obtained from a sample, and thus a separate washing procedure and the like are not required, thereby performing a more convenient and prompt detecting procedure. Therefore, the present inventors have confirmed that information about the presence or absence of a multimeric form (aggregate) and quantitative data thereof can be obtained more promptly by the method for differentially detecting a multimeric form of a multimer-forming polypeptide of the present invention, and then completed the present invention.

Accordingly, an aspect of the present invention is to provide a method for differentially detecting a multimeric form of a multimer-forming polypeptide.

Another aspect of the present invention is to provide an apparatus for differentially detecting a multimeric form of a multimer-forming polypeptide.

Other purposes and advantages of the present disclosure will become clarified by the following detailed description of the invention, claims, and drawings.

### Technical Solution

In accordance with an aspect of the present invention, there is provided a method for differentially detecting a multimeric form of a multimer-forming polypeptide in a biological sample, the method including:

(a) contacting the biological sample with an aggregation reaction inducer to induce the formation of aggregates of the multimer-forming polypeptide, the aggregation reaction inducer being a particle to which an antibody specific to the multimer-forming polypeptide is surface-bound;

(b) obtaining an image of the aggregates in step (a); and

(c) analyzing the sizes or shapes of the aggregate from the image,

wherein step (a), step (b), or steps (a) and (b) are performed on a microchip having a microchannel; wherein the analyzing of the image is performed by counting aggregates having a size greater than a reference size in a predetermined volume provided by the microchannel; wherein, when the multimeric form of the multimer-forming polypeptide is present in the biological sample, the sizes of the aggregates are greater than an aggregate of a monomeric form of the multimer-forming polypeptide; and wherein the reference size is the size of the aggregate of the monomeric form of the multimer-forming polypeptide.

The present inventors have endeavored to develop a method for differentially detecting a multimeric form of a multimer-forming polypeptide. As a result, the present inventors have developed a method for detecting an analyte by obtaining an image of an aggregation reaction material and then analyzing sizes or shapes of aggregates, unlike the method for detecting an analyte by inducing an aggregation reaction of the analyte and employing chemiluminescence-based immunoassay. The method of the present invention can detect a multimeric form of a multimer-forming polypeptide through only an image obtained from a sample, and thus a separate washing procedure and the like are not required, thereby performing a more convenient and prompt detecting procedure. Therefore, the present inventors have confirmed that information about the presence or absence of a multimeric form (aggregate) and quantitative data thereof can be obtained more promptly by the method for differentially detecting a multimeric form of a multimer-forming polypeptide of the present invention.

The present invention is intended to detect an analyte in the biological sample, particularly, an aggregate of a multimer-forming polypeptide, and is directed to a technology for obtaining information about the presence or absence of the analyte in the biological sample or quantitative data thereof by inducing an aggregation reaction using a material capable of inducing an aggregation reaction, e.g., an antibody bound to a magnetic particle, obtaining an image of an aggregation reaction material, and analyzing the occurrence or non-occurrence of the aggregation reaction and the extent of the aggregation reaction through analysis of the image.

Hereinafter, the method for differentially detecting a multimeric form of a multimer-forming polypeptide according to the present invention will be described in detail.

Step (a): Induction of Information of Aggregates of Multimer-Forming Polypeptide

First, a biological sample is contacted with an aggregation reaction inducer to induce the formation of aggregates of an analyte.

The term biological sample usable herein refers to a biological fluid. The biological sample includes preferably viruses, bacteria, tissues, cells, blood, lymph, bone marrow liquid, saliva, milk, urine, feces, ocular fluid, semen, brain homogenate, spinal fluid, synovial fluid, thymus fluid, ascites, amniotic fluid, and cell tissue fluid, more preferably tissues, cells, saliva, brain homogenate, and spinal fluid, still more preferably brain homogenate, spinal fluid, tissues, cells, and blood, and the most preferably blood, but is not limited thereto. The blood as a biological sample may be whole blood, plasma, or serum, and more preferably a plasma sample.

The aggregation reaction inducer is a particle to which an antibody specific to a multimer-forming polypeptide is surface-bound, and preferably a magnetic particle to which an antibody specifically capturing a multimer-forming polypeptide is bound. At least one antibody specific to a multimer-forming polypeptide is bound to the magnetic particle, and the sequence of an antigen binding site and the number of antibodies bound to the magnetic particle may be variously determined according to the kind of multimer-forming polypeptide and the polynucleotide sequence.

As used herein, the term “multimer-forming polypeptide” refers to a polypeptide capable of forming an aggregation form. The multimer-forming polypeptide includes amyloid-beta (Aβ) peptide, tau protein, prion, α-synuclein, Ig light chains, serum amyloid A, transthyretin, cystatin C, β2-microglobulin, huntingtin, superoxide dismutase, serpin and amylin. According to a preferable embodiment of the present invention, the multimer-forming polypeptide is amyloid-beta (Aβ) peptide.

The term aggregation reaction refers to a reaction in which a multimer-forming polypeptide binds to an antibody bound to a magnetic particle to form aggregates, and at least one antibody may be bound to the multimer-forming peptide. The antibody has a binding capacity to a multimer-forming polypeptide targeted in the biological sample. As used herein, the term “antibody” refers to an immunoglobulin protein that can be an antigen. The antibody includes an antibody fragment having binding capability to epitope, an antigen, or an antigenic fragment (e.g., F(ab′)2, Fab′, Fab, Fv) as well as a whole antibody. The antibody used herein is a polyclonal or monoclonal antibody, and preferably a monoclonal antibody.

The antibodies may be produced by the methods conventionally conducted in the art, for example, a fusion method (Kohler and Milstein, European Journal of Immunology, 6:511-519(1976)), a recombinant DNA method (U.S. Pat. No. 4,816,567), or a phage antibody library method (Clackson et al, Nature, 352:624-628(1991) and Marks et al, J. Mol. Biol., 222:58, 1-597(1991)). General procedures for the preparation of antibodies are described in detail in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, NY, 1991, which are incorporated by reference into the present specification.

In addition, a label generating a detectable signal can be selectively bound to the antibody. The label generating a detectable signal includes a chemical material (e.g., biotin), an enzymatic (e.g., alkaline phosphatase, β-galactosidase, horse radish peroxidase, and cytochrome P450), a radioactive material (e.g., C¹⁴, I¹²⁵, P³², and S³⁵), a fluorescent (e.g., fluorescein), a luminescent, a chemiluminescent, and a fluorescence resonance energy transfer (FRET), but is not limited thereto. Various labels and labeling methods are described in Ed Harlow and David Lane, Using Antibodies: A Laboratory Manual, Cold Spring Harbor Laboratory Press, 1999.

One of the characteristics of the present invention is that an antibody is allowed to bind to a surface of a solid substrate in a three-dimensional manner, thereby preparing an agglomeration reaction inducer, which is a particle to which the antibody is surface-bound. For example, a three-dimensional particle to which an antibody is bound is included in the present invention. However, the binding of an antibody to a plate surface corresponds to two-dimensional binding, and thus is excluded in the present invention. The antibody thus bound to a substrate in a three-dimensional manner is contacted with the biological sample in a three-dimensional manner, and thus has more chances to contact with the biological sample. Any material that has a three-dimensional structure may be used as the particle to which an antibody is bound, and may be preferably a material that can be easily separated or collected by gravity, charges, or magnetism.

Various materials known in the art may be used as the solid substrate. Examples of the solid substrate include polystyrene, polypropylene, glass, metal, and a hydrocarbon copolymer such as a gel. The solid substrate may be present in the form of dipstick, microtiter plate, particle (e.g., bead), affinity column, and immunoblot membrane (e.g., a polyvinylidene fluoride membrane) (see, U.S. Pat. Nos. 5,143,825, 5,374,530, 4,908,305, and 5,498,551). Preferably, the solid substrate is in the form of magnetic particles.

The magnetic particles may have various diameters depending on the size of antibody bound thereto, the kind of antibody, the number of antibodies participating in the aggregation reaction, the number of magnetic particles participating in the aggregation reaction, the temperature of the aggregation reaction, the depth and area of the microchip containing the sample, and the concentration of the sample.

Step (b): Obtainment of Image of Aggregates of Multimer-Forming Polypeptide

Next, an optical source and an imaging unit are used to obtain an image of the resultant material of step (a). Detailed descriptions of the optical source and the imaging unit are set forth below.

Step (c): Analysis of Sizes or Shapes of Aggregates

The image analysis from the image obtained in step (b) is performed by counting the aggregates according to the sizes in a predetermined volume provided by the microchannel. When the image of the multimeric form of the multimer-forming polypeptide is observed, the size of the aggregate is determined to be greater than that of an aggregate of the monomeric form control group.

The greatest characteristic of the present invention is that the aggregates of the multimer-forming polypeptide can be quantified by merely analyzing the obtained image.

According to a preferable embodiment of the present invention, the analyzing of the image is performed by counting aggregates having a size greater than a reference size in a predetermined volume provided by the microchannel.

The method according to the present invention leads to excellent sensitivity, so that the aggregates of the multimer-forming polypeptide can be differentially detected through only a trace amount of a sample. According to a preferable embodiment of the present invention, at least 1 pg/ml of a multimer of the multimer-forming polypeptide can be detected in the biological sample.

According to another aspect of the present invention, the present invention provides an apparatus for differentially detecting a multimeric form of a multimer-forming polypeptide, the apparatus including:

(a) a microchip having a microchannel for accommodating a biological sample therein;

(b) an optical source for irradiating light to the biological sample in the microchip;

(c) an imaging unit for photographing an image of the biological sample generated by the light source; and

(d) an image process for determining the presence or absence of a multimeric form of the multimer-forming polypeptide by counting aggregates having a size greater than a reference size in a predetermined volume provided by the microchannel, and processing image information about the aggregation of the multimer-forming polypeptide in the biological sample are aggregated. The reference size is the size of an aggregate of a monomeric form of the multimer-forming polypeptide.

The apparatus for differentially detecting a multimeric form of a multimer-forming polypeptide using the method of the present invention minimizes the number of floating cells in the microchannel and thus prevents the overlapping of cells, thereby accurately counting the multimeric-forms of the multimer-forming polypeptide, and can obtain information about the presence or absence of the analyte through only the analyzing of the image, thereby providing a convenient measurement method. The apparatus of the present invention uses the foregoing method for detecting a multimeric form of the multimer-forming polypeptide, and the overlapping descriptions therebetween are omitted to avoid excessive complication of the specification due to repetitive descriptions thereof. Respective components of the apparatus of the present invention will be described by steps in detail.

Component (a): Microchip Having Microchannel

The apparatus for differentially detecting a multimeric form of a multimer-forming polypeptide includes a microchip having a microchannel for accommodating a biological sample therein.

The depth of the microchip is required to be optimally designed to calculate the accurate number of multimeric forms by preventing the image overlapping between cell particles.

The microchip for accommodating a biological sample therein may further include a substrate transfer part for transferring the substrate by a predetermined distance, so that a region adjacent to an area photographed by an image unit (e.g., CCD camera) is located at a position of light incidence. Therefore, respective regions arbitrarily partitioned on the microchip may be sequentially photographed without exception. In addition, as for the apparatus using the method of the present invention, the reaction of a solid substrate to which a sample and an antibody are bound may be performed inside or outside the microchip. Therefore, the microchip may contain, preferably, a reagent, an antibody, and a solid substrate, which are used to detect the multimeric form of the multimer-forming polypeptide. The microchip is designed to automatically count the number of multimeric forms of the multimer-forming polypeptide, when the sample is dropped in the microchannel and then the microchip is mounted on the apparatus according to the present invention. Therefore, the apparatus of the present invention is easy to use and is also available for on-site diagnosis, and can be easily used by the general public as well as professionals.

The apparatus according to the present invention may further include an object lens for magnifying an image of the sample. Since the object lens enables photographing by the imaging unit (e.g., CCD camera) by magnifying the obtained image in the biological sample, the object lens is preferably located in contact with the microchip for accommodating the sample therein.

Component (b): Optical Source

As for the apparatus for differentially detecting a multimeric form of a multimer-forming polypeptide, the optical source may be selected from the group consisting of a halogen lamp, a xenon lamp, a mercury lamp, a light emitting diode, and a laser, according to the characteristics of the multimer-forming polypeptide for calculation.

The apparatus according to the present invention may further include an incident light controlling lens for controlling the quantity and focal length of light emitting from the optical source. The incident light controlling lens is disposed at the front of the optical source.

Component (c): Imaging Unit

The imaging unit included in the apparatus of the present invention photographs an image of the biological sample generated by the optical source. Various imaging units used in the art may be used, and for example, a bright field microscope, a dark field microscope, a phase-contrast microscope, a fluorescence microscope, an inverted microscope, or a CCD camera may be used. Preferably, the bright field microscope or the CCD camera is used.

Component (d): Image Processor

The image processor included in the apparatus of the present invention processes image information about the aggregation of the multimer-forming polypeptide in the biological sample from the image obtained by the imaging unit to determine the presence or absence of the multimeric form of the multimer-forming polypeptide in the biological sample and quantitative data thereof.

The image photographed by the imaging unit, e.g., a CCD camera is transmitted, and an image detection associated program is run by the image processor provided in a computer, thereby counting the number of multimeric forms of the multimer-forming polypeptide.

As described above, by using the image process according to the present invention, the number of multimeric forms of the multimer-forming polypeptide in the biological sample can be automatically counted. Particularly, the microchip for containing a biological sample therein allows the substrate to be transferred by a predetermined distance, so that a region adjacent to an area photographed by an image unit, e.g., CCD camera, is located at a position of light incidence. Therefore, respective regions arbitrarily partitioned on the substrate may be sequentially photographed. The image processor counts the multimeric forms of the multimer-forming polypeptide in the respective regions that have been sequentially photographed and then adds up the count results, thereby counting the number of multimeric forms in the entire biological sample. The number of multimeric forms of the multimer-forming polypeptide in the biological sample can be accurately and promptly determined by using this method.

The image processor counts the multimeric forms of the multimer-forming polypeptide in the respective regions that have been sequentially photographed on the substrate and then adds up the count results, so that the number of multimeric forms of the overall multimer-forming polypeptides in the biological sample can be counted. When, for example, the depth of the microchip charged with the biological sample and the area of the region photographed by the imaging unit are known, the volume of the photographed region can be obtained, and thus the volume of the biological sample containing a multimeric form of the multimer-forming polypeptide can be calculated. As such, the apparatus for differentially detecting a multimeric form of a multimer-forming polypeptide according to the present invention photographs the microchip by regions and counts multimeric forms of the multimer-forming polypeptide, thereby improving the counting accuracy. In addition, since the counting is performed on the overall regions of the biological sample even though the multimer-forming polypeptides are mal-distributed in the microchannel, the counting errors may not occur.

### Advantageous Effects

Features and advantages of the present invention are summarized as follows:

(a) The present invention is directed to a method for selectively detecting a multimer type multimer-forming polypeptide.

(b) The method of the present invention, unlike the conventional method in which an analyte is detected by chemiluminescence-based immunoassay, obtains an image of an aggregation reaction material and then analyzes sizes or shapes of aggregates, thereby capable of verifying the presence or absence of the analyte in the biological sample and quantifying the analyte.

(c) Further, the multimeric form of a multimer-forming polypeptide can be detected through only the image obtained from the sample, and thus a separate washing procedure and the like are not required, thereby performing a more convenient and prompt detecting procedure.

(d) The method for differentially detecting a multimeric form of a multimer-forming polypeptide can promptly obtain information about the presence of the multimeric form and quantitative data thereof, diagnose diseases at the early polypeptide aggregation stage, and help develop therapeutic agents directly for diseases.

### BRIEF DESCRIPTION OF THE DRAWINGS

FIG. 1 shows microscopic images of multimeric forms of the amyloid-beta protein in plasma samples of Alzheimer's patients. AD1 and AD5 represent plasma samples of Alzheimer's patients 1 and 5, respectively (magnification: 10× and 20×).

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### EXAMPLES

### Example 1

### Comparison of Data of Differential Detection

Comparison test was conducted by using the detecting method of the present invention and a method for differentially detecting a multimeric form from a monomeric form of a multimer-forming polypeptide (Korean Patent Publication No. 2010-0036324) by Peoplebio Inc.

The blood samples used herein were obtained from patients who requested examinations at an outpatient laboratory of the Department of Laboratory Medicine, Korea University Ansan Hospital, and the patient groups were randomly selected. In order to prevent blood clotting immediately after blood collection, all the blood samples were collected in a tube (BD Vacutainer USA) containing 3.2% sodium citrate. In order to obtain plasma, a general procedure for plasma collection was employed.

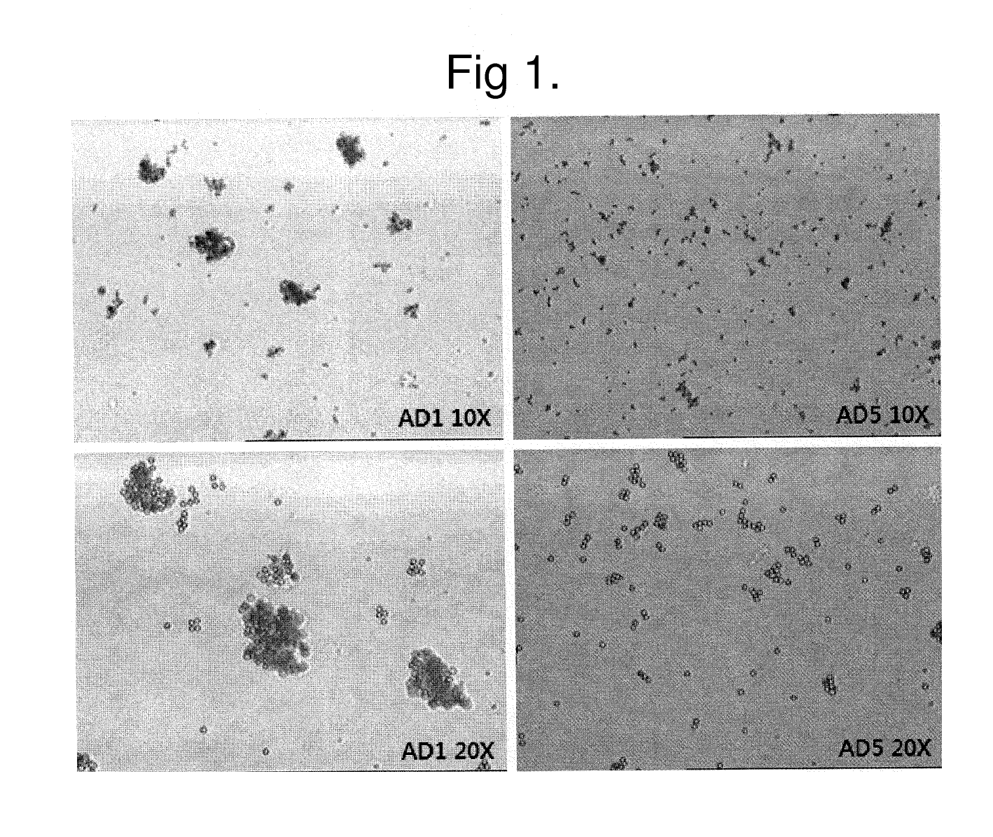

As a result of the comparison between the detecting method of the present invention and the detecting method of Peoplebio Inc., a significant correlation was shown between the results obtained by using the detecting method of the present invention and the results obtained by using the detecting method of Peoplebio Inc. As can be seen from FIG. 1, the size and the number of the multimeric forms of amyloid-beta peptide were larger and more numerous in Alzheimer's patient 1 as compared with Alzheimer's patient 5. Similarly, the relative light unit (RLU) value was higher in the sample of Alzheimer's patient 1 (Table 1).

TABLE 1
Detection results of multimeric form of multimer-
forming polypeptide by Peoplebio

| Sample | | Signal (RLU) |
|---|---|---|
| Control group | Positive | 3667051 |
| | Negative | 84542 |
| Alzheimer (AD) patient | AD-1 | 61184262 |
| | AD-5 | 534002 |
| | AD-7 | 71637372 |
| | AD-9 | 344781 |
| | AD-12 | 201123 |
| Genal (Non-AD) patient | N-1 | 132598 |
| | N-10 | 66045 |
| | N-22 | 40321 |
| | N-31 | 107074 |
| | N-36 | 70644 |

| | |
|---|---|
| Blank | 5609 |

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for differentially detecting a multimeric form of a multimer-forming polypeptide in a biological sample, the method comprising:
(a) contacting the biological sample with an aggregation reaction inducer to induce the formation of aggregates of the multimer-forming polypeptide, the aggregation reaction inducer being a particle to which an antibody specific to the multimer-forming polypeptide is surface-bound;
(b) obtaining an image of the aggregates in step (a); and
(c) analyzing the sizes or shapes of the aggregate from the image,
wherein step (a), step (b), or steps (a) and (b) are performed on a microchip having a microchannel; wherein the analyzing of the image is performed by counting aggregates having a size greater than a reference size in a predetermined volume provided by the microchannel; wherein, when the multimeric form of the multimer-forming polypeptide is present in the biological sample, the sizes of the aggregates are greater than an aggregate of a monomeric form of the multimer-forming polypeptide; and wherein the reference size is the size of the aggregate of the monomeric form of the multimer-forming polypeptide.

2. The method of claim 1, wherein the multimer-forming polypeptide is selected from the group consisting of amyloid-beta (Aβ) peptide, tau protein, prion, α-synuclein, Ig light chain, serum amyloid A, transthyretin, cystatin C, β2-microglobulin, huntingtin, superoxide dismutase, serpin and amylin.

3. The method of claim 2, wherein the multimer-forming polypeptide is amyloid-beta (Aβ) peptide.

4. The method of claim 1, wherein a label for generating a detectable signal is bound to the antibody.

5. The method of claim 1, wherein the biological sample is a biological fluid.

6. The method of claim 5, wherein the biological fluid is brain homogenate, spinal fluid, blood, plasma, or serum.

7. The method of claim 1, wherein the analyzing in step (c) is performed by counting aggregates having a size greater than the reference size from the image and then quantifying the multimeric form of the multimer-forming polypeptide from the counting results.

8. The method of claim 1, wherein at least 1 pg/ml of a multimer of the multimer-forming polypeptide is detected in the biological sample.

9. An apparatus for differentially detecting a multimeric form of a multimer-forming polypeptide, the apparatus comprising:
(a) a microchip having a microchannel for accommodating a biological sample therein;
(b) an optical source for irradiating light to the biological sample in the microchip;
(c) an imaging unit for photographing an image of the biological sample generated by the light source; and
(d) an image process for determining the presence or absence of a multimeric form of the multimer-forming polypeptide by counting aggregates having a size greater than a reference size in a predetermined volume provided by the microchannel, and processing an image information about the aggregation of the multimer-forming polypeptide in the biological sample, wherein the reference size is the size of an aggregate of a monomeric form of the multimer-forming polypeptide.

10. The apparatus of claim 9, wherein the image processor counts aggregates having a size greater than the reference size from the image and then quantifies the multimeric form of the multimer-forming polypeptide from the counting results.

11. The apparatus of claim 9, wherein at least 1 pg/ml of a multimer of the multimer-forming polypeptide is detected in the biological sample.
